(19) United States
(12) Patent Application Publication
(10) Pub. No.: US 2026/0168915 A1
(43) Pub. Date: Jun. 18, 2026

(54) TRIBOCHEMICAL IN-SITU CHARACTERIZATION DEVICE AND APPLICATION

(71) Applicants: Ningbo Institute of Materials Technology & Engineering, Chinese Academy of Sciences, NINGBO, Zhejiang Province (CN); SHAOXING GAINUO CHAOLING LUBRICATING MATERIAL CO., LTD., Shaoxing, Zhejiang Province (CN); QIANWAN INSTITUTE OF CNITECH, NINGBO, ZHEJIANG PROVINCE (CN)

(72) Inventors: HE LI, NINGBO, ZHEJIANG PROVINCE (CN); TAO CAI, NINGBO, ZHEJIANG PROVINCE (CN); WUBO CHU, NINGBO, ZHEJIANG PROVINCE (CN); BIN CHEN, SHAOXING, ZHEJIANG PROVINCE (CN); PENG SUN, NINGBO, ZHEJIANG PROVINCE (CN); BINGXUE ZHANG, NINGBO, ZHEJIANG PROVINCE (CN); NAN JIANG, NINGBO, ZHEJIANG PROVINCE (CN)

(21) Appl. No.: 18/715,342

(22) PCT Filed: Sep. 27, 2023
(86) PCT No.: PCT/CN2023/122035

§ 371 Date: May 31, 2024

(30) Foreign Application Priority Data
Sep. 6, 2023 (CN) 202311145103.2

(51) Int. Cl. G01N 19/02

(52) U.S. Cl. CPC G01N 19/02

(57) ABSTRACT

The present invention discloses a tribochemical in-situ characterization device and application. The tribochemical in-situ characterization device includes an ATR support member, an ATR crystal and a heating module, a sample groove is formed in the ATR support member, a light path channel communicated with a bottom of the sample groove is formed in the ATR support member, the ATR crystal is mounted in the light path channel, and the heating module is arranged on a lower end surface of the ATR crystal. The tribochemical in-situ characterization device further includes a mechanical friction device that is arranged in the sample groove and above the ATR crystal. The mechanical friction device includes a sliding rail fixed to a top of the ATR support member, an adapter block slidably arranged on the sliding rail and a grinding head detachably fixed to a lower end of the adapter block. The adapter block is provided with a control system, and the control system regulates and controls a pressure, a movement mode and a grinding speed of the grinding head on a surface of the ATR crystal by controlling the adapter block and collects a friction coefficient and wear rate data of a sample material. According to the present invention, lubricating grease can be continuously monitored, and a service life and a failure process of the lubricating grease can be determined.

### FIELD OF TECHNOLOGY

The present invention relates to the field of analysis and detection, in particular to a tribochemical in-situ characterization device and application.

### BACKGROUND TECHNOLOGY

Attenuated total internal reflection (ATR) accessories are a kind of test accessories widely used in the field of infrared spectrometers, which can obtain structure information of samples attached to surfaces of crystals through total internal reflection of light waves in the crystals without complicated lamination and sample preparation processes, and are a kind of convenient and practical infrared spectrometer accessories.

For example, a Chinese patent document with a publication No. CN101479638A discloses a microscope accessory configured to perform ATR measurement. The accessory has a support member that can be mounted on a movable objective table of a microscope, and a mounting member used for an ATR crystal is supported on the support member.

A Chinese patent with a publication No. CN108169186A discloses an attenuated total internal reflection spectrometer and application thereof. An accessory includes an ATR crystal component and a support member, the support member is provided with a sample groove, and the ATR crystal component includes an ATR crystal. The sample groove has an inner cavity that can accommodate a sample, and the ATR crystal is located above the inner cavity of the sample groove and connected with the sample groove.

The ATR accessories have a real-time detection or monitoring function, which can continuously or intermittently collect information of samples on surfaces of ATR crystals in real time. For example, functional group structures of media, such as lubricating grease, can be detected by the ATR crystals to determine properties of the lubricating grease. However, regarding the service performance and life evaluation of materials, such as the lubricating grease, operation is usually performed under specific conditions for a certain period of time through a friction experiment or a step experiment, and data are obtained through comparison before and after the operation. When the existing infrared spectrometer ATR accessories are used in tests, intermittent sampling is required, the ATR crystals are thoroughly cleaned after each test, operation is cumbersome, and data consistency is poor.

### SUMMARY OF THE INVENTION

In order to solve the problem of monitor consistency in tests of lubricating grease with attenuated total internal reflection (ATR) accessories, the present invention provides a tribochemical in-situ characterization device and application, which can continuously monitor the lubricating grease and determine a service life and a failure process of the lubricating grease.

A tribochemical in-situ characterization device includes an ATR support member used for a Fourier transform infrared spectrometer, an ATR crystal and a heating module, a sample groove is formed in the ATR support member, a light path channel communicated with a bottom of the sample groove is formed in the ATR support member, the ATR crystal is mounted in the light path channel, and the heating module is arranged on a lower end surface of the ATR crystal; the tribochemical in-situ characterization device further includes a mechanical friction device that is arranged in the sample groove and above the ATR crystal;

the mechanical friction device includes a horizontal sliding rail or an annular sliding rail fixed to a top of the ATR support member, an adapter block slidably arranged on the sliding rail and a grinding head detachably fixed to a lower end of the adapter block, and a material, a surface roughness and hardness parameters of the grinding head are set according to experimental requirements; the adapter block is provided with a control system, and the control system regulates and controls a pressure, a movement mode, a grinding speed and other parameters of the grinding head on a surface of the ATR crystal by controlling the adapter block and can collect a friction coefficient, a wear rate and other data of a material;

In a test process, a test sample is placed in the ATR sample groove, the sample is subjected to continuous in-situ real-time monitoring by the Fourier transform infrared spectrometer, a time origin is set when the mechanical friction device initially operates, and the pressure, the movement mode and the grinding speed are set according to experimental requirements; and changes in physical properties and chemical properties of the sample in a friction process are determined in real time according to an infrared spectrum and the friction coefficient, the wear rate and other data collected by the control system, and damage and failure time of the sample material are further determined so as to evaluate the service life of the material. Optionally, an end surface, facing the ATR crystal, of the grinding head is in a shape of round, oval or polygonal.

Optionally, the grinding head is made of stainless steel, a hard alloy, a copper alloy, an aluminum alloy, a titanium alloy, ceramic or diamond and the like.

Optionally, the ATR crystal is made of diamond, silicon, germanium, zinc selenide or monocrystalline alumina and other optical crystals.

Preferably, the sample groove has a width of 1mm-20 mm and a length of 1cm-10 cm; and the end surface, facing the ATR crystal, of the grinding head has a smaller width than the sample groove and a smaller length than the sample groove.

Preferably, the mechanical friction device is further provided with grease pulling sheets, the grease pulling sheets are arranged on two sides of the grinding head in a movement direction, and the grease pulling sheets are controlled by the control system to move up and down such that the grease pulling sheet behind the grinding head is always located in the groove for operation and lubricating grease squeezed to two sides of the sample groove by the grinding head is pulled back to a middle position, so as to avoid “empty grinding” of the grinding head due to accumulation of the lubricating grease on the two sides of the sample groove in the test process.

Preferably, the control system regulates and controls the grinding head to perform unidirectional friction or bidirectional reciprocating friction by controlling the adapter block, a friction speed is in a range of 0.01 mm/s-1,000 mm/s, and a maximum friction stroke is the length of the sample groove.

Preferably, the heating module is used for heating the sample in the sample groove, and a heating temperature in the sample groove is in a range of 0-200° C.

The present invention further provides a method for detecting indicators of lubricating grease, where an infrared spectrometer used is provided with the tribochemical in-situ characterization device, and a specific detection process includes:
coating lubricating grease to be tested in the sample groove on an upper surface of the ATR crystal;
setting an operation temperature of the lubricating grease by the heating module, regulating and controlling the pressure, the movement mode and the grinding speed of the grinding head on the surface of the ATR crystal by the control system to perform friction on the lubricating grease on the surface of the ATR crystal, and meanwhile, uploading real-time data of the pressure, the movement mode and the grinding speed to a computer by the control system;
in a friction process of the lubricating grease, irradiating infrared rays emitted by the infrared spectrometer to the lubricating grease on the upper surface of the ATR crystal through the light path channel, monitoring changes in functional group absorption peaks of the lubricating grease in the friction process in real time based on an infrared spectrum, and determining a chemical reaction of the lubricating grease in the friction process in combination with relevant data of the control system;
and monitoring changes in the friction coefficient and wear rate data of the lubricating grease on an interface by the control system, and determining service performance changes and failure time of the lubricating grease in real time in combination with the infrared spectrum.

Compared with the prior art, the present invention has the following beneficial effects.

According to the present invention, changes in functional group absorption peaks of lubricating grease in a friction process can be monitored in real time. By monitoring structural changes of the lubricating grease in the friction process, changes in physical properties and chemical properties of a sample in the friction process are determined in real time based on an infrared spectrum and data, such as the friction coefficient and the wear rate, collected by a control system, and damage and failure time of a sample material are further determined so as evaluate the service life of the sample material.

### BRIEF DESCRIPTION OF THE DRAWINGS

In order to more clearly illustrate technical solutions in examples of the present invention, drawings required to be used in description of the examples are briefly introduced below. Obviously, the drawings described below are only some examples of the present invention, and for persons of ordinary skill in the art, other drawings can also be obtained without making creative labor according to the drawings.

FIG. 1 is a structural schematic diagram of a tribochemical in-situ characterization device of the present invention;

and callouts in the figure are as follows: 1, grinding head; 2, horizontal sliding rail; 3, adapter block; 4, heating component; 5, ATR crystal; 6, ATR support member.

FIG. 2 shows an infrared spectrum of grease to be tested before friction.

FIG. 3 shows an infrared spectrum of grease to be tested after friction.

### DESCRIPTION OF THE EMBODIMENTS

The technical solutions in the examples of the present invention are clearly and completely described below in combination with the drawings attached to the examples of the present invention. Obviously, the examples described are only a part of the examples of the present invention, rather than all of the examples. Based on the examples of the present invention, all other examples obtained by persons of ordinary skill in the art without making creative labor fall within the scope of protection of the present invention.

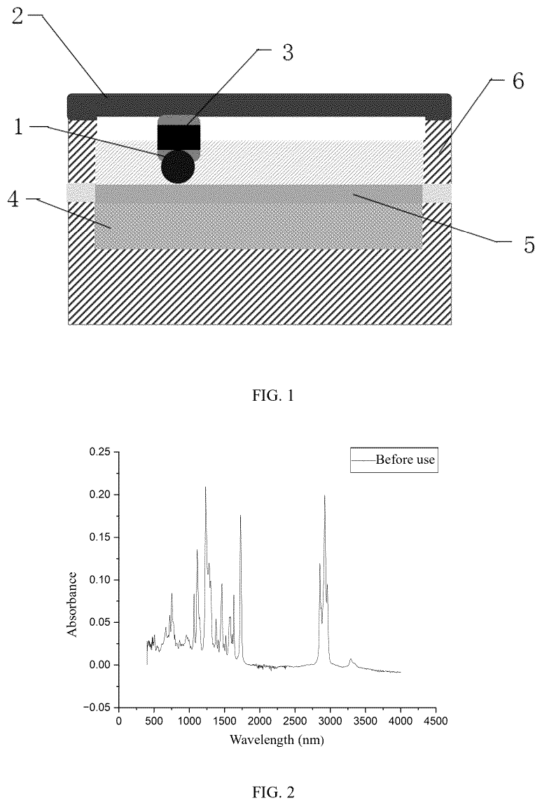

As shown in FIG. 1, a tribochemical in-situ characterization device includes an ATR support member 6 used for a Fourier transform infrared spectrometer, an ATR crystal 5 and a heating module, a sample groove is formed in the ATR support member 6, a light path channel communicated with a bottom of the sample groove is formed in the ATR support member 6, the ATR crystal 5 is mounted in the light path channel, the heating module is arranged on a lower end surface of the ATR crystal 5 and used for heating a sample in the sample groove, and a heating temperature in the sample groove is in a range of 0-200° C.

The tribochemical in-situ characterization device further includes a mechanical friction device that is arranged in the sample groove and above the ATR crystal 5. The mechanical friction device includes a sliding rail 2 (horizontal sliding rail or annular sliding rail) fixed to a top of the ATR support member 6, an adapter block 3 slidably arranged on the sliding rail 2 and a grinding head 1 detachably fixed to a lower end of the adapter block 3, and a material, a surface roughness and hardness parameters of the grinding head 1 are set according to experimental requirements. The adapter block 3 is provided with a control system, and the control system regulates and controls a pressure, a movement mode, a grinding speed and other parameters of the grinding head 1 on a surface of the ATR crystal 5 by controlling the adapter block 3 and can collect a friction coefficient, a wear rate and other data of a sample material.

The ATR crystal 5 is sealed and filled in the light path channel and hermetically cooperated with the sample groove. In an example, two ends of the light path channel are further provided with transmission window sheets, and infrared light can penetrate the transmission window sheets. The transmission window sheets are used for preventing the ATR crystal 5 from being contaminated by a foreign object, thus affecting a detection result.

Optionally, an end surface, facing the ATR crystal 5, of the grinding head 1 may be in a shape of round, oval or polygonal.

Optionally, the grinding head 1 may be made of stainless steel, a hard alloy, a copper alloy, an aluminum alloy, a titanium alloy, ceramic or diamond.

Optionally, the ATR crystal 5 may be made of diamond, silicon, germanium, zinc selenide or monocrystalline alumina.

The sample groove may have a width of 1-20 mm and a length of 1-10 cm; and the end surface, facing the ATR crystal, of the grinding head 1 has a smaller width than the sample groove and a smaller length than the sample groove.

The control system regulates and controls the grinding head 1 to perform unidirectional friction or bidirectional reciprocating friction by controlling the adapter block 3, and a friction speed and a stroke may be regulated and controlled by the control system, where the friction speed is in a range of 0.01-1,000 mm/s, the maximum friction stroke is the length of the sample groove, and a grinding path may be regulated and controlled by the control system.

Optionally, the tribochemical in-situ characterization device further includes a power supply, and the power supply is used for providing electrical energy for operation of the tribochemical in-situ characterization device.

The tribochemical in-situ characterization device of the present invention is arranged in an infrared spectrometer. A method for detecting indicators of lubricating grease includes the following steps:

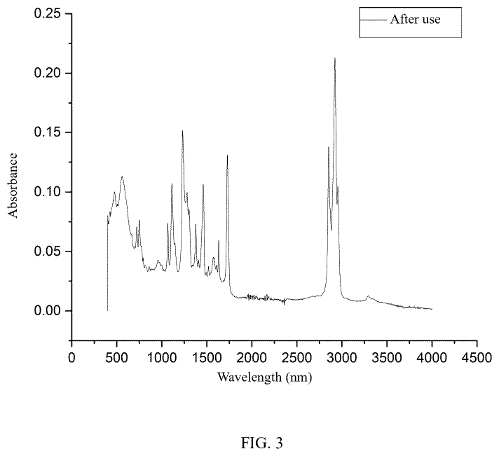

S01, coating lubricating grease to be tested in the sample groove on an upper surface of the ATR crystal, where FIG. 2 shows an infrared spectrum of the grease to be tested before friction;

S02, setting an operation temperature of the lubricating grease by the heating module, regulating and controlling the pressure, the movement mode and the grinding speed of the grinding head on the surface of the ATR crystal by the control system to perform friction on the lubricating grease on the surface of the ATR crystal, and meanwhile, uploading data of the pressure, the movement mode and the grinding speed to a computer in real time by the control system;

S03, in a friction process of the lubricating grease, irradiating infrared rays emitted by the infrared spectrometer to the lubricating grease on the upper surface of the ATR crystal through the light path channel, monitoring changes in functional group absorption peaks of the lubricating grease in the friction process in real time based on an infrared spectrum, and determining a chemical reaction of the lubricating grease in the friction process; and monitoring changes in the friction coefficient and wear rate data of the lubricating grease on an interface by the control system, and determining service performance changes and failure time of the lubricating grease in real time in combination with the infrared spectrum.

FIG. 3 shows an infrared spectrum of the grease to be tested after friction. As can be seen through comparison between FIG. 2 and FIG. 3, an in-plane bending vibration peak of C—H on a carbon skeleton of the lubricating grease is red-shifted from 1463 cm⁻¹ to 1459cm⁻¹ , indicating that the in-plane bending vibration intensity of the carbon-hydrogen bond of the lubricating grease after friction is weakened, and lubrication performance is affected. An absorption peak at 1378.34 cm⁻¹ is also red-shifted to 1377.54cm⁻¹ , also indicating that the in-plane rocking vibration of the carbon-hydrogen bond of the lubricating grease is weakened. In addition, absorption peaks at 618 cm⁻¹ and 543 cm⁻¹ are replaced by a broad peak at 560cm⁻¹ , also indicating that hydrogen on the carbon skeleton of the lubricating grease has performance loss in the friction process due to work done by a friction force.

### EXAMPLE 1

Prepared lithium-based lubricating grease was placed in the ATR sample groove, and friction performance of the lubricating grease was evaluated. A 6 mm steel ball made of GCr 15 was selected as the grinding head, and a friction test was carried out at room temperature at a load of 1 N, a reciprocating movement frequency of 1 Hz and a stroke of 10 mm, where the test was carried out for 30 minutes, and an average friction coefficient was 0.15.

### EXAMPLE 2

Prepared lithium-based lubricating grease was placed in the ATR sample groove, and friction performance of the lubricating grease was evaluated. A 6 mm small ball made of a hard alloy was selected as the grinding head, and a friction test was carried out at room temperature at a load of 1 N, a reciprocating movement frequency of 1 Hz and a stroke of 10 mm, where the test was carried out for 30 minutes, and an average friction coefficient was 0.21.

### EXAMPLE 3

Prepared lithium-based lubricating grease was placed in the ATR sample groove, and friction performance of the lubricating grease was evaluated. A 6 mm small ball made of a copper alloy was selected as the grinding head, and a friction test was carried out at room temperature at a load of 1 N, a reciprocating movement frequency of 1 Hz and a stroke of 10 mm, where the test was carried out for 30 minutes, and an average friction coefficient was 0.43.

### EXAMPLE 4

Prepared lithium-based lubricating grease was placed in the ATR sample groove, and friction performance of the lubricating grease was evaluated. A 6 mm small ball made of alumina was selected as the grinding head, and a friction test was carried out at room temperature at a load of 1 N, a reciprocating movement frequency of 1 Hz and a stroke of 10 mm, where the test was carried out for 30 minutes, and an average friction coefficient was 0.37.

### EXAMPLE 5

Prepared lithium-based lubricating grease was placed in the ATR sample groove, and friction performance of the lubricating grease was evaluated. A 6 mm small ball made of a titanium alloy was selected as the grinding head, and a friction test was carried out at room temperature at a load of 1 N, a reciprocating movement frequency of 1 Hz and a stroke of 10 mm, where the test was carried out for 30 minutes, and an average friction coefficient was 0.45.

### EXAMPLE 6

Prepared lithium-based lubricating grease was placed in the ATR sample groove, and friction performance of the lubricating grease was evaluated. A 6 mm small ball made of silicon nitride ceramic was selected as the grinding head, and a friction test was carried out at room temperature at a load of 1 N, a reciprocating movement frequency of 1 Hz and a stroke of 10 mm, where the test was carried out for 30 minutes, and an average friction coefficient was 0.54.

### EXAMPLE 7

Prepared lithium-based lubricating grease was placed in the ATR sample groove, and friction performance of the lubricating grease was evaluated. A 6 mm steel ball made of GCr 15 was selected as the grinding head, and a friction test was carried out at room temperature at a load of 5 N, a reciprocating movement frequency of 1 Hz and a stroke of 10 mm, where the test was carried out for 30 minutes, and an average friction coefficient was 0.12.

### EXAMPLE 8

Prepared lithium-based lubricating grease was placed in the ATR sample groove, and friction performance of the lubricating grease was evaluated. A 6 mm steel ball made of GCr 15 was selected as the grinding head, and a friction test was carried out at room temperature at a load of 10 N, a reciprocating movement frequency of 1 Hz and a stroke of 10 mm, where the test was carried out for 30 minutes, and an average friction coefficient was 0.18.

### EXAMPLE 9

Prepared lithium-based lubricating grease was placed in the ATR sample groove, and friction performance of the lubricating grease was evaluated. A 6 mm steel ball made of GCr 15 was selected as the grinding head, and a friction test was carried out at room temperature at a load of 5 N, a reciprocating movement frequency of 3 Hz and a stroke of 10 mm, where the test was carried out for 30 minutes, and an average friction coefficient was 0.13.

### EXAMPLE 10

Prepared lithium-based lubricating grease was placed in the ATR sample groove, and friction performance of the lubricating grease was evaluated. A 6 mm steel ball made of GCr 15 was selected as the grinding head, and a friction test was carried out at room temperature at a load of 5 N, a reciprocating movement frequency of 7 Hz and a stroke of 10 mm, where the test was carried out for 30 minutes, and an average friction coefficient was 0.16.

### EXAMPLE 11

Prepared lithium-based lubricating grease was placed in the ATR sample groove, and friction performance of the lubricating grease was evaluated. A 6 mm steel ball made of GCr 15 was selected as the grinding head, and a friction test was carried out at room temperature at a load of 5 N, a reciprocating movement frequency of 10 Hz and a stroke of 10 mm, where the test was carried out for 30 minutes, and an average friction coefficient was 0.22.

The technical solutions and beneficial effects of the present invention are described in detail through the above examples. It is understood that the above descriptions are merely specific examples of the present invention and are not intended to limit the present invention. Any modifications, supplements and equivalent substitutions made within the scope of principles of the present invention shall be included within the scope of protection of the present invention.

1. A tribochemical in-situ characterization device, comprising an attenuated total internal reflection (ATR) support member used for a Fourier transform infrared spectrometer, an ATR crystal and a heating module, wherein a sample groove is formed in the ATR support member, a light path channel communicated with a bottom of the sample groove is formed in the ATR support member, the ATR crystal is mounted in the light path channel, the heating module is arranged on a lower end surface of the ATR crystal, and the tribochemical in-situ characterization device further comprises a mechanical friction device that is arranged in the sample groove and above the ATR crystal;
the mechanical friction device comprises a sliding rail fixed to a top of the ATR support member, an adapter block slidably arranged on the sliding rail and a grinding head detachably fixed to a lower end of the adapter block, and a material, a surface roughness and hardness parameters of the grinding head are set according to experimental requirements; the adapter block is provided with a control system, and the control system regulates and controls a pressure, a movement mode and a grinding speed of the grinding head on a surface of the ATR crystal by controlling the adapter block and collects a friction coefficient and wear rate data of a sample material;
in a test process, a test sample is placed in the ATR sample groove, the sample is subjected to continuous in-situ real-time monitoring by the Fourier transform infrared spectrometer, a time origin is set when the mechanical friction device initially operates, and the pressure, the movement mode and the grinding speed are set according to experimental requirements; and changes in physical properties and chemical properties of the sample in a friction process are determined in real time according to an infrared spectrum and the friction coefficient and the wear rate data collected by the control system, and damage and failure time of the sample material are further determined so as to evaluate the service life of the sample material.

2. The tribochemical in-situ characterization device according to claim 1, wherein an end surface, facing the ATR crystal, of the grinding head is in a shape of round, oval or polygonal.

3. The tribochemical in-situ characterization device according to claim 1, wherein the grinding head is made of stainless steel, a hard alloy, a copper alloy, an aluminum alloy, a titanium alloy, ceramic or diamond.

4. The tribochemical in-situ characterization device according to claim 1, wherein the ATR crystal is made of diamond, silicon, germanium, zinc selenide or monocrystalline alumina.

5. The tribochemical in-situ characterization device according to claim 1, wherein the sample groove has a width of 1mm-20 mm and a length of 1cm-10 cm;
and the end surface, facing the ATR crystal, of the grinding head has a smaller width than the sample groove and a smaller length than the sample groove.

6. The tribochemical in-situ characterization device according to claim 1, wherein the control system regulates and controls the grinding head to perform unidirectional friction or bidirectional reciprocating friction by controlling the adapter block, a friction speed is in a range of 0.01 mm/s-1,000 mm/s, and a maximum friction stroke is the length of the sample groove.

7. The tribochemical in-situ characterization device according to claim 1, wherein the heating module is used for heating the sample in the sample groove, and a heating temperature in the sample groove is in a range of 0-200° C.

8. The tribochemical in-situ characterization device according to claim 1, wherein the mechanical friction device further comprises grease pulling sheets, the grease pulling sheets are arranged on two sides of the grinding head in a movement direction, and the grease pulling sheets are controlled by the control system to move up and down such that the grease pulling sheet behind the grinding head is always located in the groove for operation and lubricating grease squeezed to two sides of the sample groove by the grinding head is pulled back to a middle position, so as to avoid empty grinding of the grinding head due to accumulation of the lubricating grease on the two sides of the sample groove in the test process.

9. A method for detecting indicators of lubricating grease, wherein an infrared spectrometer used is provided with the tribochemical in-situ characterization device according to claim 1, and a specific detection process comprises:
coating lubricating grease to be tested in the sample groove on an upper surface of the ATR crystal;
setting an operation temperature of the lubricating grease by the heating module, regulating and controlling the pressure, the movement mode and the grinding speed of the grinding head on the surface of the ATR crystal by the control system to perform friction on the lubricating grease on the surface of the ATR crystal, and meanwhile, uploading data of the pressure, the movement mode and the grinding speed to a computer in real time by the control system;
in a friction process of the lubricating grease, irradiating infrared rays emitted by the infrared spectrometer to the lubricating grease on the upper surface of the ATR crystal through the light path channel, monitoring changes in functional group absorption peaks of the lubricating grease in the friction process in real time based on an infrared spectrum, and determining a chemical reaction of the lubricating grease in the friction process;
and monitoring changes in the friction coefficient and wear rate data of the lubricating grease on an interface by the control system, and determining service performance changes and failure time of the lubricating grease in real time in combination with the infrared spectrum.
